(19) United States
(12) Patent Application Publication
(10) Pub. No.: US 2015/0093744 A1
(43) Pub. Date: Apr. 2, 2015

(54) CAPTURE OF HUMAN NOROVIRUS FROM CLINICAL ENVIRONMENTAL AND FOOD SAMPLES AND MEASUREMENT OF INFECTIVITY

(71) Applicant: The United States of America, as represented by the Secretary of Agriculture, Washington, DC (US)

(72) Inventors: Peng Tian, Hercules, CA (US); Depeng Wang, Shanghai (CN)

(21) Appl. No.: 14/503,162

(22) Filed: Sep. 30, 2014

Related U.S. Application Data
(60) Provisional application No. 61/884,949, filed on Sep. 30, 2013.

(51) Int. Cl. C12Q 1/70

(52) U.S. Cl. CPC C12Q 1/70
USPC 435/5

(57) ABSTRACT

The present invention relates to human noroviruses.

### REFERENCE TO RELATED APPLICATIONS

This application claims priority to U.S. Provisional Patent Application Ser. No. 61/884,949, filed Sep. 30, 2013, which is incorporated herein by reference in its entirety.

### FIELD OF THE INVENTION

The invention relates to detection and quantitation of infectious Human norovirus.

### BACKGROUND OF THE INVENTION

Norovirus is a genus of genetically diverse single-stranded RNA, non-enveloped viruses in the Caliciviridae family. The viruses are highly contagious and are transmitted by fecally contaminated food or water; by person-to-person contact; and via aerosolization of the virus and subsequent contamination of surfaces. Noroviruses are the most common cause of viral gastroenteritis in humans, typically accounting for approximately 60% of food-borne illnesses in the United States (Scallan et al., 2011). Although Human noroviruses (HuNoV) are pathogens of significant concern, they currently cannot be cultured, which limits the ways in which they can be studied.

Molecular-based virus quantitation assays, notably qRT-PCR, have been used in lieu of in vivo/ex vivo assays for a number of reasons, including e.g., speed, ease, and feasibility. It is, however, questionable that what they quantitate is any accurate reflection of actual virus activity. Prior de-activation assays using Tulane Virus (TV), a cultureable analogue of HuNoV, have shown that there is little-to-no-correlation between virus counts obtained from TCID50 (50% Tissue Culture Infective Dose) assays as compared to virus signal obtained from the qRT-PCR assay. While qRT-PCR is effective at quantitating the integrity of the targeted amplicon, it cannot detect critical damage to other areas of the viral genome outside of the qRT-PCR target amplicon, and most notably, it cannot detect critical degradation of the capsid required for binding and infection. Therefore qRT-PCR is only capable of detecting a subset of de-activating injuries/degradations to the virus, and its quantitation is inflated by false positives from otherwise de-activated/degraded virions.

Because HuNoVs are highly contagious viruses with important human health effects what is needed in the art are methods for accurately detecting and quantitating HuNoVs. Fortunately, as will be clear from the following disclosure, the present invention provides for these and other needs.

### SUMMARY OF THE INVENTION

In one exemplary embodiment, the disclosure provides a method for detecting infectious capsid-enveloped viral particles in a biological sample, the method comprising: (i) attaching an viral receptor to a container substrate thereby providing a container affixed capture agent; (ii) adding the biological sample to the container affixed capture agent; (iii) incubating the biological sample with the container affixed capture agent thereby capturing infectious capsid-enveloped viral particles as receptor-binding-intact capsids and providing receptor-bound-intact-capsids, wherein the receptor-bound-intact-capsids contain viral genomic nucleic acids; (iv) adding PCR reagents to the receptor-bound-intact-capsids; (v) amplifying the viral genomic nucleic acids contained within the receptor-bound-intact capsids with the PCR reagents, thereby providing amplified viral genomic nucleic acids as PCR products; (vi) analyzing the amplified viral genomic nucleic acids to determine the presence and quantity of infectious capsid-enveloped viral particles; thereby detecting infectious capsid-enveloped viral particles in a biological sample. In one exemplary embodiment, the capsid-enveloped viral particles are members selected from the group consisting of human Norovirus and Tulane virus. In another exemplary embodiment, the receptor is a member selected from the group consisting of Human Blood Group Antigen (HBGA), porcine gastric mucin (PGM) and specific antibodies.

In one exemplary embodiment, the disclosure provides a method for detecting infectious human Norovirus (HuNoV) in a biological sample, the method comprising: (i) attaching an HuNoV receptor to a container substrate thereby providing a container affixed capture agent; (ii) adding the biological sample to the container affixed capture agent; (iii) incubating the biological sample with the container affixed capture agent thereby capturing infectious HuNoV as receptor-binding-intact capsids and providing receptor-bound-intact-capsids, wherein the receptor-bound-intact-capsids contain HuNoV genomic nucleic acids; (iv) adding PCR reagents to the receptor-bound-intact-capsids; (v) amplifying the HuNoV genomic nucleic acids contained within the receptor-bound-intact capsids with the PCR reagents, thereby providing amplified HuNoV genomic nucleic acids as PCR products; (vi) analyzing the amplified HuNoV genomic nucleic acids to determine the presence and quantity of infectious HuNoV; thereby detecting infectious HuNoV in a biological sample. In one exemplary embodiment, the biological sample is a member selected from the group consisting of a clinical sample, an environmental sample and a food sample or a combination of said members. In another exemplary embodiment, the HuNoV receptor is a member selected from the group consisting of Human Blood Group Antigen (HBGA) and porcine gastric mucin (PGM). In another exemplary embodiment, the container substrate is a microtiter plate. In one exemplary embodiment, the method is used to confirm that a sample has been disinfected of HuNoV.

In some exemplary embodiments, the method is automated.

Other features, objects and advantages of the invention will be apparent from the detailed description which follows.

### BRIEF DESCRIPTION OF THE DRAWINGS

FIG. 1. Recovery of Tulane Virus (TV) with various loading doses and with or without heat release of viral RNA. TV copy numbers were shown in Y-axis in log and loading dose was shown in X-axis.

FIG. 2 Binding of viral capsid to HBGA receptor measured by ELISA (A) and damage in viral genome measured by long (1.6 Kb) and short (0.5 Kb) RT-PCRs (B). A. OD405 was shown in Y-axis. Inactivation conditions were indicated in X-axis where 72 C and 56 C stands for treatment at 72° C. and 56° C. for 2 min.; UV600, and 60 stands for treatment with UV at 600 and 60 mJ/cm²; CL600, and 300 stands for chlorine treatment at 600 and 300 ppm; E70 and E50 stands for treatment at 70% and 50% EtOH for 20 s; PC and NC stands for positive and negative controls. B. Inactivation conditions were indicated in X-axis where PC stands for positive control for heat and UV and cPC stands for positive control for chlorine.

FIG. 3 Inactivation of HuNoV by thermal treatment. HuNoV copy numbers in log (Y-axis) after treatment with elevated temperatures of 56, 63, 72, and 100° C. for 2 min (A); after treated at 72° C. for up to 5 min (B); or treated at 63° C. up to 60 min (C).

FIG. 4. Inactivation of HuNoV by chlorine. HuNoV copy numbers in log (Y-axis) after treatment with free chlorine at 2, 4, 8, 16, and 32 ppm for 10 min.

FIG. 5 Inactivation of HuNoV by UV. HuNoV copy numbers in log (Y-axis) after treatment with UV-C radiation at 250, 500, 750, 1000, and 1500 mJ/cm².

### DETAILED DESCRIPTION OF THE INVENTION

### Definitions

All technical and scientific terms used herein, unless otherwise defined below, are intended to have the same meaning as commonly understood by one of ordinary skill in the art; references to techniques employed herein are intended to refer to the techniques as commonly understood in the art, including variations on those techniques or substitutions of equivalent techniques which would be apparent to one of skill in the art. In order to more clearly and concisely describe the subject matter disclosed herein, the following definitions are provided for certain terms which are used in the specification and appended claims.

The term “prevent” or “prevention” as used herein, refers to any indica of success in prevention or amelioration of disease or infection, including any objective or subjective parameter such as abatement, remission, and/or diminishing of symptoms. For example, the terms “prevent” or “prevention” as used herein, refer to the prevention of disease associated with Human Noroviruses; reduction in the severity of disease associated with Human Noroviruses; reduction in expected deaths etc. The prevention, treatment, reduction or amelioration of symptoms can be based on objective or subjective parameters; including the results of physical examination, biopsy or microscopic examination of a tissue sample, or any other appropriate means known in the art.

The expression “histo-blood group antigen”, “human histo-blood group antigen or “HGBA” as used herein, refers to complex carbohydrates on red blood cells, mucosal epithelia, saliva, milk and other body fluids, which are highly polymorphic and are related to the ABO, secretor and Lewis antigens, in which carbohydrate core structures, constitute antigenically distinct phenotypes. As is well known in the art, HuNoVs rely on recognition of human histo-blood group antigens (HBGAs) as ligands or receptors for attachment (see e.g., Huang, P., J. Infect. Dis. 188:19-31)

The term “sample” as used herein refers to a set of objects e.g., human norovirus (HuNoV) particles in their milieu, from a parent population that includes all such objects in a particular environment e.g., a sample of HuNoV taken from one sub-area of a larger surface. In general, a “sample” as used herein refers to an unbiased (representative) sample comprising a subset of objects chosen from a complete population using a selection process that does not depend on the properties of the objects. Thus, determining the properties of a “sample” will reflect the properties of the complete population.

The term “biological sample” or the term “diagnostic sample” as used herein, refers to any sample obtained from food, surfaces or living or dead organisms.

The term “TCID50” refers to the 50% Tissue Culture Infective Dose which is a measure of infectious virus titer. “TCID50” is known in the art (see e.g., Flint, S. J.; et al. (2009). “Virological Methods”. Principles of Virology. ASM Press).

The expression “container substrate” as used herein, refers to a substrate to which HuNoV receptors can be bound which is also capable of functioning as a container suitable for use as a container for carrying out PCR or other amplification reaction. HuNoV receptors can be bound by any method known in the art. In an exemplary embodiment, a container substrate is a microtiter plate. In other exemplary embodiments, a container substrate is a petri plate. In still other exemplary embodiments, a container substrate is a microfuge tube or test tube.

The terms “isolated,” “purified,” or “biologically pure” as used herein, refer to material that is substantially or essentially free from components that normally accompany it as found in its native state. In an exemplary embodiment, purity and homogeneity are determined using analytical chemistry techniques such as polyacrylamide gel electrophoresis or high performance liquid chromatography. A nucleic acid that is the predominant species present in a preparation is substantially purified. In one exemplary embodiment, an isolated Human Norovirus nucleic acid is separated from open reading frames and/or other nucleic acid sequences that flank the bacteriphage lytic enzyme nucleic acid in its native state. Similarly, an isolated Human Norovirus is isolated from components which accompany it in a biological sample. In an exemplary embodiment, the term “purified” denotes that a nucleic acid or protein gives rise to essentially one band in an electrophoretic gel. Typically, isolated nucleic acids or proteins have a level of purity expressed as a range. The lower end of the range of purity for the component is about 60%, about 70% or about 80% and the upper end of the range of purity is about 70%, about 80%, about 90% or more than about 90%.

The term “nucleic acid” as used herein, refers to a polymer of ribonucleotides or deoxyribonucleotides. Typically, “nucleic acid” polymers occur in either single- or double-stranded form, but are also known to form structures comprising three or more strands. The term “nucleic acid” includes naturally occurring nucleic acid polymers as well as nucleic acids comprising known nucleotide analogs or modified backbone residues or linkages, which are synthetic, naturally occurring, and non-naturally occurring, which have similar binding properties as the reference nucleic acid, and which are metabolized in a manner similar to the reference nucleotides. Examplary analogs include, without limitation, phosphorothioates, phosphoramidates, methyl phosphonates, chiral-methyl phosphonates, 2-O-methyl ribonucleotides, peptide-nucleic acids (PNAs).

Unless otherwise indicated, a particular nucleic acid sequence also implicitly encompasses conservatively modified variants thereof (e.g., degenerate codon substitutions) and complementary sequences, as well as the sequence explicitly indicated. Specifically, degenerate codon substitutions may be achieved by generating sequences in which the third position of one or more selected (or all) codons is substituted with mixed-base and/or deoxyinosine residues (see e.g., Batzer et al., Nucleic Acid Res. 19:5081 (1991); Ohtsuka et al., J. Biol. Chem. 260:2605-2608 (1985); and Rossolini et al., Mol. Cell. Probes 8:91-98 (1994)).

The terms “polypeptide,” “peptide” and “protein” are used interchangeably herein to refer to a polymer of amino acid residues. The terms apply to naturally occurring amino acid polymers and non-naturally occurring amino acid polymers as well as amino acid polymers in which one or more amino acid residues is an artificial chemical mimetic of a corresponding naturally occurring amino acid.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refers to compounds that have the same basic chemical structure as a naturally occurring amino acid, i.e., an a carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions in a manner similar to a naturally occurring amino acid.

Amino acids are referred to herein by either their commonly known three letter symbols or by the one-letter symbols recommended by the IUPAC-IUB Biochemical Nomenclature Commission. Nucleotides, likewise, may be referred to by their commonly accepted single-letter codes.

“Conservatively modified variants” applies to both amino acid and nucleic acid sequences. With respect to particular nucleic acid sequences, conservatively modified variants refers to those nucleic acids which encode identical or essentially identical amino acid sequences, or where the nucleic acid does not encode an amino acid sequence, to essentially identical sequences. Because of the degeneracy of the genetic code, a large number of functionally identical nucleic acids encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variations. Every nucleic acid sequence herein which encodes a polypeptide also describes every possible silent variation of the nucleic acid. One of skill will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid which encodes a polypeptide is implicit in each described sequence.

As to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art (see, e.g., Creighton, Proteins (1984)). Such conservatively modified variants are in addition to and do not exclude polymorphic variants, interspecies homologs, and alleles.

The following eight groups illustrate some exemplary amino acids that are conservative substitutions for one another:
1) Alanine (A), Glycine (G);
2) Aspartic acid (D), Glutamic acid (E);
3) Asparagine (N), Glutamine (Q);
4) Arginine (R), Lysine (K);
5) Isoleucine (1), Leucine (L), Methionine (M), Valine (V);
6) Phenylalanine (F), Tyrosine (Y), Tryptophan (W);
7) Serine (S), Threonine (T); and
8) Cysteine (C), Methionine (M)

Macromolecular structures such as polypeptide structures are described in terms of various levels of organization. For a general discussion of this organization, see, e.g., Alberts et al., Molecular Biology of the Cell (3^{rd} ed., 1994) and Cantor and Schimmel, Biophysical Chemistry Part I: The Conformation of Biological Macromolecules (1980). “Primary structure” refers to the amino acid sequence of a particular peptide. “Secondary structure” refers to locally ordered, three dimensional structures within a polypeptide. These structures are commonly known as domains. Domains are portions of a polypeptide that form a compact unit of the polypeptide and are typically 50 to 350 amino acids long. Typical domains are made up of sections of lesser organization such as stretches of β-sheet and α-helices. “Tertiary structure” refers to the complete three dimensional structure of a polypeptide monomer. “Quaternary structure” refers to the three dimensional structure formed by the noncovalent association of independent tertiary units. Anisotropic terms are also known as energy terms.

The term “label” as used herein, refers to a composition detectable by spectroscopic, photochemical, biochemical, immunochemical, or chemical means. Exemplary labels include ³²P, fluorescent dyes, electron-dense reagents, enzymes (e.g., as commonly used in an ELISA), biotin, digoxigenin, or haptens and proteins for which antisera or monoclonal antibodies are available.

As used herein a “nucleic acid probe or oligonucleotide” refers to a nucleic acid capable of binding to a target nucleic acid of complementary sequence through one or more types of chemical bonds, usually through complementary base pairing, usually through hydrogen bond formation. As used herein, a probe may include natural (i.e., A, G, C, or T) or modified bases (e.g., 7-deazaguanosine, inosine, etc.). In addition, the bases in a probe may be joined by a linkage other than a phosphodiester bond, so long as it does not interfere with hybridization. Thus, for example, probes may be peptide nucleic acids in which the constituent bases are joined by peptide bonds rather than phosphodiester linkages. It will be understood by one of skill in the art that probes may bind target sequences lacking complete complementarity with the probe sequence depending upon the stringency of the hybridization conditions. In one exemplary embodiment, probes are directly labeled as with isotopes, chromophores, lumiphores, chromogens etc. In other exemplary embodiments probes are indirectly labeled e.g., with biotin to which a streptavidin complex may later bind. By assaying for the presence or absence of the probe, one can detect the presence or absence of the select sequence or subsequence.

Thus, the term “labeled nucleic acid probe or oligonucleotide” as used herein refers to a probe that is bound, either covalently, through a linker or a chemical bond, or noncovalently, through ionic, van der Waals, electrostatic, or hydrogen bonds to a label such that the presence of the probe may be detected by detecting the presence of the label bound to the probe.

The term “primer” as used herein, refers to short nucleic acids, typically DNA oligonucleotides of at least about 15 nucleotides in length. In an exemplary embodiment, primers are annealed to a complementary target DNA strand by nucleic acid hybridization to form a hybrid between the primer and the target DNA strand. Annealed primers are then extended along the target DNA strand by a DNA polymerase enzyme. Primer pairs can be used for amplification of a nucleic acid sequence, e.g., by the polymerase chain reaction (PCR) or other nucleic-acid amplification methods known in the art.

PCR primer pairs are typically derived from a known sequence, for example, by using computer programs intended for that purpose such as Primer (Version 0.5 © 1991, Whitehead Institute for Biomedical Research, Cambridge, Mass.). One of skill in the art will appreciate that the specificity of a particular probe or primer increases with its length. Thus, for example, a primer comprising 20 consecutive nucleotides of a Clostridium perfringens bacteriophage lytic enzyme sequence will anneal to a related target sequence with a higher specificity than a corresponding primer of only 15 nucleotides. Thus, in an exemplary embodiment, greater specificity of a nucleic acid primer or probe, is attained with probes and primers selected to comprise 20, 25, 30, 35, 40, 50 or more consecutive nucleotides of a selected sequence.

Nucleic acid probes and primers are readily prepared based on the nucleic acid sequences disclosed herein. Methods for preparing and using probes and primers and for labeling and guidance in the choice of labels appropriate for various purposes are discussed, e.g., in Sambrook et al., Molecular Cloning, A Laboratory Manual 3rd ed. 2000, Cold Spring Harbor Laboratory; and Current Protocols in Molecular Biology, Ausubel et al., eds., 1994, John Wiley & Sons). The term “recombinant” when used with reference, e.g., to a cell, or nucleic acid, protein, or vector, indicates that the cell, nucleic acid, protein or vector, has been modified by the introduction of a heterologous nucleic acid or protein or the alteration of a native nucleic acid or protein, or that the cell is derived from a cell so modified. Thus, for example, recombinant cells express genes that are not found within the native (non-recombinant) form of the cell or express native genes that are otherwise abnormally expressed, over expressed, under expressed or not expressed at all.

The term “capable of hybridizing under stringent hybridization conditions” as used herein, refers to annealing a first nucleic acid to a second nucleic acid under stringent hybridization conditions (defined below). In an exemplary embodiment, the first nucleic acid is a test sample, and the second nucleic acid is the sense or antisense strand of a Clostridium perfringens bacteriophage lytic enzyme. Hybridization of the first and second nucleic acids is conducted under standard stringent conditions, e.g., high temperature and/or low salt content, which tend to disfavor hybridization of dissimilar nucleotide sequences.

The term “operably linked” refers to a functional linkage between a nucleic acid expression control sequence (such as e.g., an array of transcription factor binding sites) and a second nucleic acid sequence (such as e.g., Human Norovirus nucleic acid sequence), wherein the expression control sequence directs expression e.g., transcription, of the nucleic acid corresponding to the second sequence. In an exemplary embodiment, a promoter that is “operably linked” to a heterologous nucleic acid e.g., a Clostridium perfringens bacteriophage lytic enzyme, is located upstream of and in-frame with the heterologous nucleic acid.

The term “heterologous” when used with reference to portions of a nucleic acid indicates that the nucleic acid comprises two or more subsequences that are not found in the same relationship to each other in nature. For instance, the nucleic acid is typically recombinantly produced, having two or more sequences from unrelated genes arranged to make a new functional nucleic acid, e.g., a promoter from one source and a coding region from another source. Similarly, a heterologous protein indicates that the protein comprises two or more subsequences that are not found in the same relationship to each other in nature (e.g., a fusion protein).

The following terms are used to describe the sequence relationships between two or more nucleic acids or polynucleotides: “reference sequence”, “comparison window”, “sequence identity”, “percentage of sequence identity”, and “substantial identity”. A “reference sequence” is a defined sequence used as a basis for a sequence comparison; a reference sequence may be a subset of a larger sequence, for example, as a segment of a full-length Clostridium perfringens bacteriophage lytic enzyme sequence or gene sequence given in a sequence listing, or may comprise a complete Clostridium perfringens bacteriophage lytic enzyme sequence or gene sequence.

The terms “identical” or percent “identity,” in the context of two or more nucleic acids or polypeptide sequences, refer to two or more sequences or subsequences that are the same or have a specified percentage of amino acid residues or nucleotides that are the same (e.g., 85% identity, 90% identity, 99%, or 100% identity), when compared and aligned for maximum correspondence over a comparison window, or designated region as measured using a sequence comparison algorithm or by manual alignment and visual inspection.

The phrase “substantially identical”, in the context of two nucleic acids or polypeptides, refers to two or more sequences or subsequences that have at least about 85%, identity, at least about 86%, 87%, 88%, 89%, 90%, 91%, 92%, 93%, 94%, 95%, 96%, 97%, 98%, 99%, or 100% nucleotide or amino acid residue identity, when compared and aligned for maximum correspondence, as measured using a sequence comparison algorithm or by visual inspection. In an exemplary embodiment, the substantial identity exists over a region of the sequences that is at least about 50 residues in length. In another exemplary embodiment, the substantial identity exists over a region of the sequences that is at least about 100 residues in length. In still another exemplary embodiment, the substantial identity exists over a region of the sequences that is at least about 150 residues or more, in length. In one exemplary embodiment, the sequences are substantially identical over the entire length of nucleic acid or protein sequence.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters.

A “comparison window”, as used herein, includes reference to a segment of any one of the number of contiguous positions selected from the group consisting of from 20 to 600, usually about 50 to about 200, more usually about 100 to about 150 in which a sequence may be compared to a reference sequence of the same number of contiguous positions after the two sequences are optimally aligned. Methods of alignment of sequences for comparison are well-known in the art. Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by manual alignment and visual inspection (see, e.g., Current Protocols in Molecular Biology (Ausubel et al., eds. 1995 supplement)).

An exemplary algorithm for sequence comparison is PILEUP. PILEUP creates a multiple sequence alignment from a group of related sequences using progressive, pairwise alignments to show relationship and percent sequence identity. It also plots a tree or dendogram showing the clustering relationships used to create the alignment. PILEUP uses a simplification of the progressive alignment method of Feng & Doolittle, J. Mol. Evol. 35:351-360 (1987). The method used is similar to the method described by Higgins & Sharp, CABIOS 5:151-153 (1989). The program can align up to 300 sequences, each of a maximum length of 5,000 nucleotides or amino acids. The multiple alignment procedure begins with the pairwise alignment of the two most similar sequences, producing a cluster of two aligned sequences. This cluster is then aligned to the next most related sequence or cluster of aligned sequences. Two clusters of sequences are aligned by a simple extension of the pairwise alignment of two individual sequences. The final alignment is achieved by a series of progressive, pairwise alignments. The program is run by designating specific sequences and their amino acid or nucleotide coordinates for regions of sequence comparison and by designating the program parameters. Using PILEUP, a reference sequence is compared to other test sequences to determine the percent sequence identity relationship using the following parameters: default gap weight (3.00), default gap length weight (0.10), and weighted end gaps. PILEUP can be obtained from the GCG sequence analysis software package, e.g., version 7.0 (Devereaux et al., Nuc. Acids Res. 12:387-395 (1984).

Another example of algorithm that is suitable for determining percent sequence identity and sequence similarity are the BLAST and BLAST 2.0 algorithms, which are described in Altschul et al., Nuc. Acids Res. 25:3389-3402 (1977) and Altschul et al., J. Mol. Biol. 215:403-410 (1990), respectively. Software for performing BLAST analyses is publicly available through the National Center for Biotechnology Information (http://www.ncbi.nlm.nih.gov/). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) or 10, M=5, N4 and a comparison of both strands. For amino acid sequences, the BLASTP program uses as defaults a wordlength of 3, and expectation (E) of 10, and the BLOSUM62 scoring matrix (see Henikoff & Henikoff, Proc. Natl. Acad. Sci. USA 89:10915 (1989)) alignments (B) of 50, expectation (E) of 10, M=5, N=4, and a comparison of both strands.

The BLAST algorithm also performs a statistical analysis of the similarity between two sequences (see, e.g., Karlin & Altschul, Proc. Nat'l. Acad. Sci. USA 90:5873-5787 (1993)). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.2, more preferably less than about 0.01, and most preferably less than about 0.001.

An indication that two nucleic acid sequences or polypeptides are substantially identical is that the polypeptide encoded by the first nucleic acid is immunologically cross reactive with the antibodies raised against the polypeptide encoded by the second nucleic acid, as described below. Thus, a polypeptide is typically substantially identical to a second polypeptide, for example, where the two peptides differ only by conservative substitutions. Another indication that two nucleic acid sequences are substantially identical is that the two molecules or their complements hybridize to each other under stringent conditions, as described below. Yet another indication that two nucleic acid sequences are substantially identical is that the same primers can be used to amplify the sequence.

The phrase “selectively (or specifically) hybridizes to” refers to the binding, duplexing, or hybridizing of a molecule only to a particular nucleotide sequence under stringent hybridization conditions when that sequence is present in a complex mixture (e.g., total cellular or library DNA or RNA). In general, two nucleic acid sequences are said to be “substantially identical” when the two molecules or their complements selectively or specifically hybridize to each other under stringent conditions.

The phrase “stringent hybridization conditions” refers to conditions under which a probe will hybridize to its target subsequence, typically in a complex mixture of nucleic acid, but to no other sequences. Stringent conditions are sequence-dependent and will be different in different circumstances. Longer sequences hybridize specifically at higher temperatures. An extensive guide to the hybridization of nucleic acids is found in Tijssen, Techniques in Biochemistry and Molecular Biology-Hybridization with Nucleic Probes, “Overview of principles of hybridization and the strategy of nucleic acid assays” (1993). Generally, stringent conditions are selected to be about 5-10° C. lower than the thermal melting point (Tₘ) for the specific sequence at a defined ionic strength pH. The Tₘ is the temperature (under defined ionic strength, pH, and nucleic concentration) at which 50% of the probes complementary to the target hybridize to the target sequence at equilibrium (as the target sequences are present in excess, at Tₘ, 50% of the probes are occupied at equilibrium). Stringent conditions will be those in which the salt concentration is less than about 1.0 M sodium ion, typically about 0.01 to 1.0 M sodium ion concentration (or other salts) at pH 7.0 to 8.3 and the temperature is at least about 30° C. for short probes (e.g., 10 to 50 nucleotides) and at least about 60° C. for long probes (e.g., greater than 50 nucleotides). Stringent conditions may also be achieved with the addition of destabilizing agents such as formamide. For high stringency hybridization, a positive signal is at least two times background, preferably 10 times background hybridization. Exemplary high stringency or stringent hybridization conditions include: 50% formamide, 5×SSC and 1% SDS incubated at 42° C. or 5×SSC and 1% SDS incubated at 65° C., with a wash in 0.2×SSC and 0.1% SDS at 65° C. However, other high stringency hybridization conditions known in the art can be used.

Nucleic acids that do not hybridize to each other under stringent conditions are still substantially identical if the polypeptides that they encode are substantially identical. This occurs, for example, when a copy of a nucleic acid is created using the maximum codon degeneracy permitted by the genetic code. In such cases, the nucleic acids typically hybridize under moderately stringent hybridization conditions. Exemplary “moderately stringent hybridization conditions” include a hybridization in a buffer of 40% formamide, 1 M NaCl, 1% SDS at 37° C., and a wash in 1×SSC at 45° C. A positive hybridization is at least twice background. Those of ordinary skill will readily recognize that alternative hybridization and wash conditions can be utilized to provide conditions of similar stringency.

### I. Introduction:

Human noroviruses (HuNoVs) are the major cause of epidemic non-bacterial gastroenteritis. Unfortunately, due to the inability to cultivate HuNoVs, it has been a challenge to determine their infectivity. Quantitative real-time RT-PCR (qRT-PCR) is widely used in detecting HuNoVs. However, qRT-PCR only detects the presence of viral RNA and does not indicate viral infectivity. Indeed, a virus could lose its infectivity by damage to its viral capsid, but the viral RNA may still persist to be detected by qRT-PCR. Likewise, even if a treatment can lethally-damage the viral RNA, that damage may occur at one-or-more regions outside of the short span of the targeted qRT-PCR amplicon, so that too may still persist to be detected by qRT-PCR. Thus, for the purpose of detecting viable and infectious HuNoVs, conventional qRT-PCR is hindered by these false positives.

Human blood group antigens (HBGAs) have been identified previously as receptors for human and some animal NoVs. The method disclosed herein employs HBGAs as a container-affixed capture agent that serves both to concentrate virus from solution, as well as that to select the receptor-binding-intact capsids. As will be apparent from the disclosure provided hereinbelow, HBGA-cqRT-PCR method disclosed herein effectively concentrates HuNoV and thus permits one to distinguish the inactivated virus from infectious virus.

### II. Isolating Human Norovirus and Constructing Expression Vectors

A. General Methods

This invention utilizes routine techniques in the field of recombinant genetics. Basic texts disclosing the general methods of use in this invention include Sambrook et al., Molecular Cloning—A Laboratory Manual (3rd Ed.), Vol. 1-3, Cold Spring Harbor Laboratory, Cold Spring Harbor, N.Y., 2000; Kriegler, Gene Transfer and Expression: A Laboratory Manual (1990); and Current Protocols in Molecular Biology (Ausubel et al., eds., 1994)). Unless otherwise noted, technical terms are used according to conventional usage. Definitions of common terms in molecular biology maybe found in e.g., Benjamin Lewin, Genes V, published by Oxford University Press, 1994 (ISBN 0-19-854287-9); Kendrew et al. (eds.), The Encyclopedia of Molecular Biology, published by Blackwell Science Ltd., 1994 (ISBN 0-632-02182-9); and Robert A. Meyers (ed.), Molecular Biology and Biotechnology: a Comprehensive Desk Reference, published by VCH Publishers, Inc., 1995 (ISBN 1-56081-569-8).

For nucleic acids, sizes are given in either kilobases (kb) or base pairs (bp). Estimates are typically derived from agarose or acrylamide gel electrophoresis, from sequenced nucleic acids, or from published DNA sequences. For proteins, sizes are given in kilodaltons (kDa) or amino acid residue numbers. Proteins sizes are estimated from gel electrophoresis, from sequenced proteins, from derived amino acid sequences, or from published protein sequences.

Oligonucleotides that are not commercially available can be chemically synthesized e.g., according to the solid phase phosphoramidite triester method first described by Beaucage & Caruthers, Tetrahedron Letts. 22:1859-1862 (1981), using an automated synthesizer, as described in Van Devanter et al., Nucleic Acids Res. 12:6159-6168 (1984). Purification of oligonucleotides is by either native acrylamide gel electrophoresis or by anion-exchange HPLC as described in Pearson & Reanier, J. Chrom. 255:137-149 (1983).

The sequence of the cloned genes and synthetic oligonucleotides can be verified after cloning using, e.g., the chain termination method for sequencing double-stranded templates of Wallace et al., Gene 16:21-26 (1981).

Methods disclosed herein may also utilize routine techniques in the field of microbiology. Basic texts disclosing the general methods of use in this invention include, e.g., Methods for General and Molecular Microbiology, 3rd Edition, C. A. Reddy, et al., eds. ASM Press (2008); and Encyclopedia of Microbiology, 2nd ed., Joshua Lederburg, ed., Academic Press (2000).

Unless otherwise noted, technical terms are used according to conventional usage. Definitions of common terms in microbiology maybe found in e.g., Microbiology By Cliffs Notes, I. Edward Alcamo, Wiley (1996); Encyclopedia of Microbiology, (2000) supra; Singleton et al., Dictionary of Microbiology and Molecular Biology (2d ed. 1994). Definitions of common terms in molecular biology maybe found in e.g., Benjamin Lewin, Genes V, published by Oxford University Press, 1994 (ISBN 0-19-854287-9); Kendrew et al. (eds.), The Encyclopedia of Molecular Biology, published by Blackwell Science Ltd., 1994 (ISBN 0-632-02182-9); and Robert A. Meyers (ed.), Molecular Biology and Biotechnology: a Comprehensive Desk Reference, published by VCH Publishers, Inc., 1995 (ISBN 1-56081-569-8).

B. Methods for the Isolation of Nucleic Acids Comprising Human Norovirus Sequences

Human norovirus nucleic acids can be isolated using any of a variety of methods known to those of skill in the art which may be used for isolation of viral nucleic acid sequences. For example, Human norovirus nucleic acids can be isolated from viral particles using e.g, RNA extraction methods e.g., using Qiagen RNA Extraction kit.

Other methods known to those of skill in the art can also be used to isolate RNA comprising HuNoVs nucleic acid sequences. See e.g., Sambrook, et al. for a description of other techniques for the isolation of RNAs related to RNA molecules of known sequence.

Kits

In an exemplary embodiment, kits comprising at least one microtiter plate coated with human Blood Group antigens or other HuNoV receptor, primers for detecting HuNoV and written instructions for using the kit to detect HuNoV in biological samples.

The following examples are offered to illustrate, but not to limit the invention.

### EXAMPLES

### Example 1

The following example illustrates that the capture methods disclosed herein using in situ quanitative real time PCR (ISC-qRT-PCR) are effective for distinguishing infectivity of Tulane virus (TV).

Similar to HuNoV, the recently-discovered Tulane virus (TV) recognizes the type A and B HBGAs as receptors for infection (see e.g., Farkas et al., (2010) J Virol 84(17), 8617-25). Accordingly, as the following Example illustrates, we used Tulane Virus as a “surrogate” for HuNoV to show that ISC-qRT-PCR is an effective method for distinguishing infectivity of the virus, by comparing it side-by-side against a tissue-culture-based assay.

### Materials and Methods for Example 1

### Virus Cultivation.

LLC-MK2 cells (American Type Culture Collection, Manassas, Va.) were used for culturing and titration of virus (Wei et al., (2008) J Virol. 82:11429-11436). Tulane virus was kindly provided by Dr. Jiang (Division of Infectious Diseases, Cincinnati Children's Hospital Medical Center, Cincinnati Ohio). Cells were grown in HyClone M199/EBSS medium (Thermo Fisher Scientific, Waltham, Mass.) supplemented with 10% fetal bovine serum (Atlanta Biologicals, Norcross, Ga.) and 2× Gibco antibiotic-antimycotic (Life Technologies, Carlsbad, Calif.). One day after plating cells, confluent cell cultures were inoculated with virus at a multiplicity of infection (MOI) of 0.1, and then harvested 2 days post-inoculation by scraping. The cultures were then transferred to sterile 50-ml centrifuge tubes (Becton Dickinson and Company, Franklin Lakes, N.J.) and freeze-thawed (cycles of −20° C. and 25° C.) three times to release cell-associated viruses. Cellular debris was removed by centrifugation at 2,500 RCF for 10 min and the virus-enriched supernatant was aliquotted and stored at −20° C.

### Virus Titering by TCID50 Assay.

The virus stock was titered using the 50% tissue culture infective dose (TCID50) assay (see e.g., Tian et al., (2013) J Food Prot 76(4), 712-8). One hundred microliters of serially-diluted virus stock ranging from 10⁻¹ to 10⁻⁵ was added to each quantization well in a 96-well plate, organized with each serial dilution occupying a row, at 10 quantization wells/row. The plate was incubated for 5-6 days, and then examined for cell balling via microscopy. Balling counts were converted into TCID50 and TCID50/ml using a Reed & Muench Calculator spreadsheet (see e.g., Reed, L. J.; Muench, H. (1938). “A simple method of estimating fifty percent endpoints”. The American Journal of Hygiene 27: 493-497). Similarly, viable virus counts after treatment with experimental conditions (described above) were also determined by TCID50 assay.

### Rapid Amplification-qRT-PCR Assay (RA-qRT-PCR).

One hundred microliters of treated (or control) TV was added into each well in 6-well plate. After 60 min incubation at 37° C. for virus attachment, the unbound virus was removed and the well was washed 3 times with 3 ml of culture media. One milliliter of culture media was then added into each well, and the cells were incubated 37° C. for 24 hours. Cells were frozen-then-thawed three times, extracted for viral RNA using a Qiagen viral RNA extraction kit, and then quantiated for viral RNA described as follows. The RA-qRT-PCR method is similar to TCID50 assay but faster.

Virus RNA Extraction and Quantitative Real-Time RT-PCR (qRT-PCR).

One hundred microliters of each treated (and control) virus was extracted for RNA with a Qiagen viral RNA extraction kit (Qiagen USA, Valencia, Calif.) in accordance with the manufacturer's protocol. Extracted viral RNA was quantitated with probe-based quantitative real-time RT-PCR using a qPCR system (“MX3000P”, Stratagene; La Jolla, Calif.) with a one-step qRT-PCR kit (“Quantitect Probe RTPCR Kit”, Qiagen USA; Valencia, Calif.) in accordance with the manufacturer's protocol. The primers and probes used for detection of TV were: TV forward (5′ TGA CGA TGA CCT TGC GTG 3′ SEQ ID NO:1), TV reverse (5′ TGG GAT TCA ACC ATG ATA CAG TC 3′ SEQ ID NO:2), TV probe (5′ HEX-ACC CCA AAG CCC CAG AGT TGA T-BHQ-1 3′ SEQ ID NO:3). Each 25.0 μL reaction consisted of 12.5 μL of Quantitect Probe RT-PCR master mix, 7.5 μL of RNAse-free water, 0.75 μL of each primer (TV forward, TV reverse, both at 10 μM), 0.25 μL of TV probe at 10 μM, 0.25 μL of Quantitect RT mix, and 3.0 μL of extracted RNA. Cycling times and temperatures were 50° C. for 30 min, 95° C. for 15 min, followed by 45 cycles of 95° C. for 15 sec, 53° C. for 20 sec, and 60° C. for 50 sec (see e.g., Tian et al., 2013, supra).

### In Situ Capture-qRT-PCR (ISC-qRT-PCR).

Human saliva was collected from three blood-type B volunteers and mixed. The aggregated saliva was boiled for 5 min. and then centrifuged at 10,000 RCF for another 5 min. The clarified supernatant was aliquotted and stored at −20° C. until use. Nunc Top-Yield Module (VWR, Brisbane, Calif.) wells were coated with 100 μl of a 1:1,000 dilution (using 0.5M carbonate-bicarbonate buffer, pH 9.6) of clarified saliva at 4° C. overnight, then blocked with 1% BSA in PBS at 37° C. for 1 hour. The wells were washed with PBS and either used immediately or stored at 4° C. for near-future use. One hundred microliters of TV was added to each well, incubated at 37° C. for 30 min, and then washed three times with PBS to clear any unbound viruses. Ten microliters of RNase-free dH₂O was added to each well, which is then sealed with polyolefin sealing film (VWR, West Chester, Pa., USA), brought to 95° C. for 5 min, then cooled to 4° C. To this heat-released RNA in solution, 12.5 μl of a prepared qRT-PCR master-mix was added, and the wells are then re-sealed with polyolefin sealing film. The qRT-PCR master-mix contains qRT-PCR buffer, mix of primers (300 nM), probe (100 nM), and reverse-transcriptase/polymerase (2.5 U). Cycling times and temperatures were 50° C. for 30 min, 95° C. for 15 min, followed by 42 cycles of 95° C. for 15 sec, 53° C. for 20 sec, and 60° C. for 50 s.

### Partial and Full Inactivation Conditions for TV.

1. Thermal Inactivation

Three-hundred microliter aliquots of virus stock in 1.5 ml microcentrifuge tubes were incubated in 56° C. and 72° C. heat blocks for 2 min and quickly cooled on ice. The control samples were kept at room temperature.

2. Chlorine Inactivation

Bleach (Pure Bright, Conord, Ontario, Canada) was diluted to make fresh chlorine stock. The neutralizer reagent, Sodium thiosulfate (Fisher Scientific, Waltham, Mass.), was also made fresh at the same concentration. Three-hundred microliter aliquots of virus stock in 1.5 mL microcentrifuge tubes were brought to experimental free chlorine conditions (300 or 600 ppm) and allowed to incubate for 10 min at room temperature. The free chlorine treatment conditions were neutralized by the addition of an equal amount (relative to the chlorine solution added) of sodium thiosulfate solution. Non-treatment, control reactions were set-up in parallel without chlorine.

3. EtOH Inactivation

Three-hundred microliter aliquots of virus stock in 1.5 ml microcentrifuge tubes were diluted with 100% EtOH to arrive at EtOH treatment concentrations of 40% and 70%, and allowed to incubate for 20 sec at room temperature. The incubation is immediately quenched afterwards by 10-fold dilution with MEM to lower the ethanol concentration and to reduce cytotoxicity. Aliquots of virus were also diluted with MEM at corresponding concentrations (40% and 70%) for untreated controls.

4. UV Irradiation

Three-hundred microliters aliquots of virus stock were placed into a 90 mm Petri dishes and were UV-irradiated with energies of 30 mJ/cm² and 60 mJ/cm² using a Stratagene UV cross-linker (Stratalinker 1800, emits UV-C at 254 nm at ˜3 milliwatts/cm2). Non-treatment, control reactions were set-up in parallel without UV-irradiation.

### ELISA Assays

Mouse anti-TV antibodies (kindly provided by Dr. Jiang) were used in a “sandwich” ELISA assay similar to one described previously (Tian et al., (2010) Journal of Applied Microbiology, 109 pp. 1753-1762). Briefly, Nunc Top-Yield Module (VWR, Brisbane, Calif.) wells were coated with a 1:1000 dilution of aggregated saliva from blood-type-B individuals (in 0.5M carbonate-bicarbonate buffer, pH 9.6) and incubated at 4° C. overnight, then blocked with bovine serum albumin (BSA, 1.0% in PBS) at 37° C. for 1 hour. One hundred microliters of treated or untreated TV was added into each well and incubated at 37° C. for 30 min. After three washes with PBS, anti-rTV antibody (diluted 1:3,000 in PBS, 100 μl/well) was added to TV-bound wells and incubated at 37° C. for 30 min. After three washes with PBS, the bound anti-rTV antibodies were detected by the addition of alkaline phosphatase (AP)-conjugated rabbit anti-mouse IgG antibodies (Zymed Laboratories, South San Francisco, Calif.; diluted 1:3000 in PBS, 100 μl/well), followed by development with p-nitrophenyl phosphate substrate (1 mg/ml, 100 μl/well). Negative controls included PGM-coated modules without the addition of TV; and BSA-blocked modules without PGM coating, but with 100 μl/well of TV. The average optical density from a set of experimental wells was divided by the average optical density of all negative-control wells to obtain a value designated “P/N”.

### Long and Short RT-PCR.

Primer P885F (5′-TTGGCTCAACACTGTGCAAAAG-3′SEQ ID NO:4) and P886R (5′-CTGATTGCATTTTCCAAACAGC-3′SEQ ID NO:5) were used to amply a short fragment (523 bp) in the viral genome located from 1214 to 1716 (Farkas et al., 2008). Primer TV-ORF2F (5′-ATGGAAAACAGCAAAACTGAAC-3′SEQ ID NO:6) and TV-ORF2R (5′-TTATCTAAAGACAACTGCTGTC-3′SEQ ID NO:7) were used for amplification of a long fragment (1,605 bp) in the viral genome located from 4380 to 5984. One-step qRT-PCR kit was used for RT-PCR reaction. Cycling times and temperatures were 50° C. for 30 min, 95° C. for 15 min, followed by 32 cycles of 94° C. for 30 sec, 52° C. for 30 sec, and 72° C. for 2 min and a final extension of 5 min at 72° C. for long RT-PCR; and 50° C. for 30 min, 95° C. for 15 min, followed by 32 cycles of 94° C. for 30 sec, 53° C. for 30 sec, and 72° C. for 1 min, and a final extension of 5 min at 72° C. for short RT-PCR, respectively. PCR product was analyzed on 1% agarose gels in the presence of ethidium bromide.

### Data Analysis and Statistics.

Each plating experiment was repeated three times (N=3) as independent replicates with triplicates in each experiment (n=3). One-way ANOVA was employed for data analysis.

### Results for Example 1

### Optimal Conditions and Sensitivity of ISC-qRT-PCR Method.

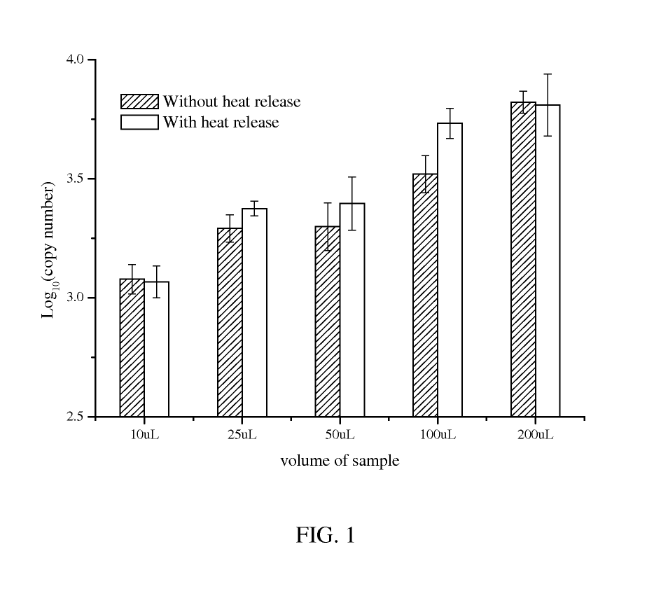

A dose-dependent binding was observed from loading dose of 10 to 200 μl/well of the virus (FIG. 1). Although the signal from loading dose of 200 μl per well was still higher than the signal obtained from loading dose of 100 μl, it was not significantly different and the increase was limited. Therefore, the loading dose for the rest experiment in this study was set to 100 μl. The viral RNA signals from receptor-captured-virus were significantly higher with the inclusion of heat-denaturation than that without heat-denaturation in all doses except for loading dose of 200 μl. This illustrated the effectiveness and importance of releasing the viral RNA from the captured viruses by heat-denaturation prior to qRT-PCR. Therefore for the rest of this study, all samples were heat-denatured prior to qRT-PCR. The sensitivity of the optimized ISC-qRT-PCR was compared with the qRT-PCR with RNA extracted by commercial RNA extraction kit (Table 1). The results suggested that the ISC-qRT-PCR provides an equal result than qRT-PCR using extracted viral RNA.

TABLE 1

| | Measured by qRT-PCR with | |
|---|---|---|
| | extracted viral RNA copy | Measured by ISC-qRT-PCR |
| Dilution | number(copies) | copy number (copies) |
| 10⁻¹ | 2199 ± 191 | 1083 ± 291 |
| 10⁻² | 292 ± 33 | 144 ± 15 |
| 10⁻³ | 41 ± 4 | 26 ± 2 |
| 10⁻⁴ | 0.6 | 0.6 |
| 10⁻⁵ | 0.6 | 0.6 |

Inactivation of TV Measured by ISC-qRT-PCR and Amplification Based qRT-PCR Methods.

The infectivity of TV under partial and full-inactivation conditions was measured by three methods (Table 2).

TABLE 2

| | Treatment | | | | | | | | |
|---|---|---|---|---|---|---|---|---|---|
| | Temperature | | Chlorine | | Ultraviolet | | | | |
| Detection | (° C.) | | (ppm) | | (mJ/cm²) | | EtOH | | Positive |
| method | 56 | 72 | 300 | 600 | 30 | 60 | 40% | 70% | control |
| INC-qRT-PCR | 2.7 ± 0.1 | −0.2 ± 0 | 2.2 ± 0.0 | −0.2 ± 0 | 4.0 ± 0.1 | 3.8 ± 0.2 | 3.1 ± 0.1 | −0.2 ± 0 | 4.4 ± 0.1 |
| (copy number) | (555) | (0.6) | (148) | (0.4) | (9,873) | (6,635) | (1,215) | (0.4) | (23,285) |
| Amplification | 3.8 ± 0.1 | −0.2 ± 0 | 3.5 ± 0.1 | −0.2 ± 0 | 4.1 ± 0.1 | −0.2 ± 0 | 2.6 ± 0.2 | 2.3 ± 0.2 | 4.6 ± 0.0 |
| assay | (6442) | (0.4) | (3411) | (0.4) | (13710) | (0.4) | (419) | (187) | (40,133) |
| (copy number) | | | | | | | | | |
| TCID₅₀ | 4.6 ± 0.1 | 0 | 2.8 ± 0.1 | 0 | 2.7 ± 0.1 | 0 | 4.7 ± 0.2 | 0 | 5.4 ± 0.1 |
| (copy number) | (40,600) | (0) | (704) | (0) | (518) | (0) | (46,600) | (0) | (257,000) |

ISC-qRT-PCR quantitation of TV partially and fully-inactivated with heat, chlorine and EtOH treatments could be correlated to the quantitation results of two tissue-culture-based assays. After treatment of TV with heat or chlorine at full inactivation conditions, neither virus genome nor amplification of virus was detectable by the ISC-qRT-PCR method nor by tissue-culture-based assays (RA-qRT-PCR and TCID50). After treatment of TV with EtOH at full inactivation conditions, neither virus genome nor amplification of virus was detectable by the ISC-qRT-PCR method and by TCID50, while RA-qRT-PCR quantitated an over 99.5% reduction of TV titer. After treatment of TV with heat at partial inactivation conditions, the reduction in genomic copy or virus titer was 97.6%, 83.9%, and 69.3% as measured by ISC-qRT-PCR, RA-qRT-PCR, and TCID50, respectively. After treatment of TV with chlorine at partial inactivation conditions, the reduction in genomic copy or virus titer was 99.4%, 91.5%, and 99.7% as measured by ISC-qRT-PCR, RA-qRT-PCR, and TCID50, respectively. After treatment of TV with 40% EtOH, the reduction in genomic copy or virus titer was 94.8%, 99.0%, and 81.9% as measured by ISC-qRT-PCR, RA-qRT-PCR, and TCID50, respectively. In contrast to the general correlation seen for the other treatments, ISC-qRT-PCR detection of viral inactivation differed significantly from that of the tissue-culture-based assays. After treatment of TV with UV at full inactivation conditions (60 mJ/cm²), ISC-qRT-PCR only quantified a 71.5% reduction in genomic copy, while both RA-qRT-PCR and TCID50 quantified a 100% reduction.

### Binding of Viral Capsid to Human Blood Group Antigen (HBGA) Receptor Measured by ELISA and Damage in Viral Genome Measured by Long or Short Amplicon RT-PCR Assays.

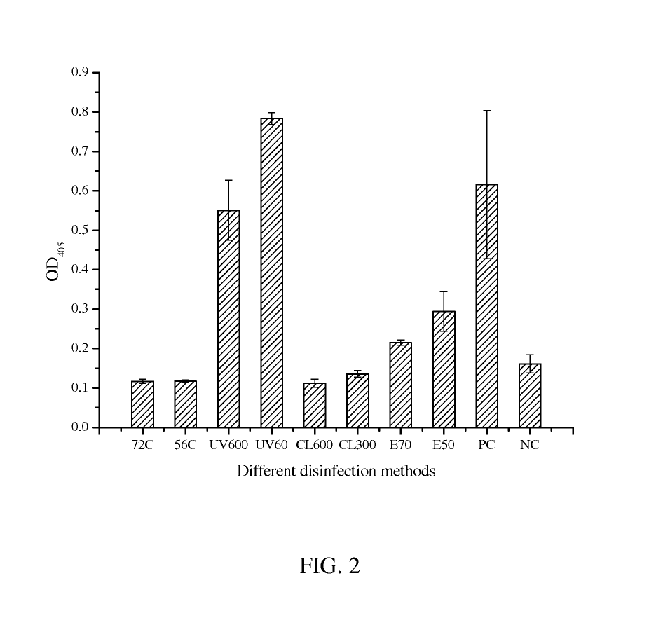

Binding of viral capsid to Human Blood Group Antigen (HBGA) was significantly reduced under partial or full inactivation conditions by heat, chlorine, or EtOH. The results suggest that the primary mechanism of viral inactivation by these three treatments was that of alteration and/or denaturation of capsid proteins, which results in failure of binding to the receptor. Heat and chlorine treatment was more effective at viral inactivation than EtOH. Unlike the other treatments, enhanced binding was occurred when TV was exposed to partial inactivation (data not shown) or full inactivation dose of UV (FIG. 2A). A UV irradiation dose as high as 600 mJ/cm2 resulted in no significant reduction in binding ability to HBGA. There was no significant reduction in the amplification of a short amplicon (0.5 Kb) within the viral RNA as measured by qRT-PCR under all inactivation treatments and levels (FIG. 2B). A longer amplicon within the viral genome (1.6 Kb) could not be amplified at all after full inactivation treatments by heat and chlorine, or by UV irradiation at a dose ten times higher than the inactivation dose required by TCID50. This suggests that the mechanism of viral inactivation by UV irradiation is not at the level of interaction between virus capsid and its receptor.

### Discussion for Example 1

Norovirus is an extremely infectious virus, but one that can not be cultured, as it lacks both a tissue culture system and small animal models. Although molecular approaches such as RT-PCR and quantitative real time RT-PCR (qRT-PCR) have been developed and utilized for the detection of HuNoV, they are unable to distinguish the infectivity of the virus. A virus could lose its infectivity by damage to its viral capsid, but the viral RNA may still persist to be detected by qRT-PCR.

Similar to HuNoV, TV recognizes type A and B HBGAs as receptors for infection (Farkas et al., 2010, supra), and thus, can serve as an improved surrogate for HuNoV in the respect of interaction between virus and its receptor. We demonstrated that there was a good correlation between infectious status measured by ISC-qRT-PCR and cell-culture-based amplification assays (TCID50 and amplification assay) for TV inactivated by heat, chlorine and EtOH. The ISC-qRT-PCR method could be used to evaluate virus inactivated by damaging the capsid structure or interaction between the capsid and virus receptor. We demonstrated that heat, chlorine, and EtOH treatment primarily cause changes in capsid structure and binding to viral receptor. Therefore, inactivation of TV by these methods could be correctly reflected by the ISC-qRT-PCR method. As TV and HuNoV uses the same receptor for infection, this ISC-qRT-PCR method provides useful information on inactivation conditions for HuNoV.

### Example 2

In the following Example, we demonstrated that the ISC-qRT-PCT method is effectively used to evaluate infectivity of HuNoV. Full inactivation of HuNoV was defined as greater than 3 logs of reduction in viral copy numbers in this study. We demonstrated that ISC-qRT-PCR method could be used as a new method to distinguish the inactivated HuNoV from infectious virus.

### Materials and Methods for Example 2

### Preparation of HuNoV Samples

A GII.4 NoV was used in the study (Tian et al., 2010, supra). The virus sample was diluted (1:10) into buffer (PBS pH 7.2). The stool suspension was clarified of gross solids by low-speed centrifugation (2000 RCF for 20 minutes), and the supernatant was then filtered through a 0.45 μM nylon membrane (Autovial Syringeless Filters; Whatman plc, Maidstone, Kent, UK).

### Preparation of HBGA-Coated Modules for In Situ Capture-qRT-PCR (ISC-qRT-PCR).

Type III porcine gastric mucin (PGM) purified from porcine stomach mucosa was purchased from Sigma (St. Louis, Mich.; cat. no. M-1778). Wells of the Nunc Top-Yield Module (VWR, Brisbane, Calif.) were coated with 100 μl of PGM (1 mg/ml in 0.5M carbonate-bicarbonate buffer, pH 9.6) at 4° C. overnight, and blocked with 1% BSA in PBS at 37° C. for 1 hour. The wells were washed with PBS and used immediately or kept at 4° C. for future use.

### In Situ Capture-qRT-PCR (IS C-qRT-PCR).

One hundred μL of PBS-diluted HuNoV was added to each well of the module and incubated at 37° C. for 30 min. After three washes with PBS, 10 μL RNase free dH₂O was added to each well, sealed with polyolefin sealing tape (VWR, West Chester, Pa., USA) and heated at 95° C. for 5 min, followed by cooling at 4° C. 12.5 μL of qRT-PCR master-mix containing primers (10 nM), probe (10 nM) and reverse-transcriptase/polymerase (0.25 U) were added to each well and re-sealed with polyolefin sealing tape. The qRT-PCR was performed in a MX3000P real-time PCR system (Stratagene) using the following amplification protocol: RT reaction at 50° C. for 30 min, RT denaturation at 95° C. for 15 min; PCR amplification for 42 cycles consisting of denaturation at 94° C. for 15 s, annealing at 53° C. for 20 s, and extension at 60° C. for 50 s.

### Thermal Inactivation of HuNoV.

Three-hundred microliter aliquots of virus stock in 1.5 ml microcentrifuge tubes were heat-treated in heat blocks at 56, 63, 72, and 100° C. for periods of time ranging from 1 to 60 min (Tian et al., 2013, supra), and then quickly cooled in an ice bath. 100 μl of treated or untreated virus was used in each ISC-qRT-PCR assay.

### Chlorine Inactivation of HuNoV.

Bleach (Pure Bright, Conord, Ontario, Canada) was used to make fresh chlorine stock. The neutralizing reagent, sodium thiosulfate (Fisher Scientific, Waltham, Mass.), was also made freshly at the same concentration (Tian et al., 2013, supra). Three-hundred microliters aliquots of virus stock in 1.5 mL microcentrifuge tubes were adjusted to experimental free chlorine levels (0-32 ppm), quickly vortexed and allowed to incubate for 10 min. The free chlorine treatment conditions were then neutralized by equal amounts of sodium thiosulfate solution. 100 μl of treated or untreated virus was used in each ISC-qRT-PCR assay.

### EtOH Inactivation of HuNoV.

Fifteen microliter aliquots of virus stock in 1.5 ml micro-centrifuge tubes were diluted with 35 μl 100% EtOH to arrive at an EtOH treatment concentration of 70%, and then allowed to incubate for 20 s at room temperature (Duizer et al., 2004). A similar 15 μl aliquot of virus was instead diluted with 35 μl PBS for untreated controls. 100 μl of EtOH treated or PBS treated virus was used in ISC-qRT-PCR assay.

### UV Irradiation Inactivation of HuNoV.

Three-hundred and twenty microliters aliquots of virus stock were placed into 90 mm Petri dishes, and were UV-irradiated with energies of 250, 500, 750, 1,000, and 1,500 mJ/cm2 using a Stratagene UV Stratalinker 1800 (emitted as UV-C at 254 nm at ˜3 milliwatts/cm2). 100 μl of treated or untreated virus was used in ISC-qRT-PCR assay.

### Data Analysis and Statistics.

Each plating experiment was repeated three times (N=3) as independent replicates with triplicates in each experiment (n=3). One-way ANOVA was employed for data analysis.

### Results for Example 2

### Thermal Inactivation of HuNoV

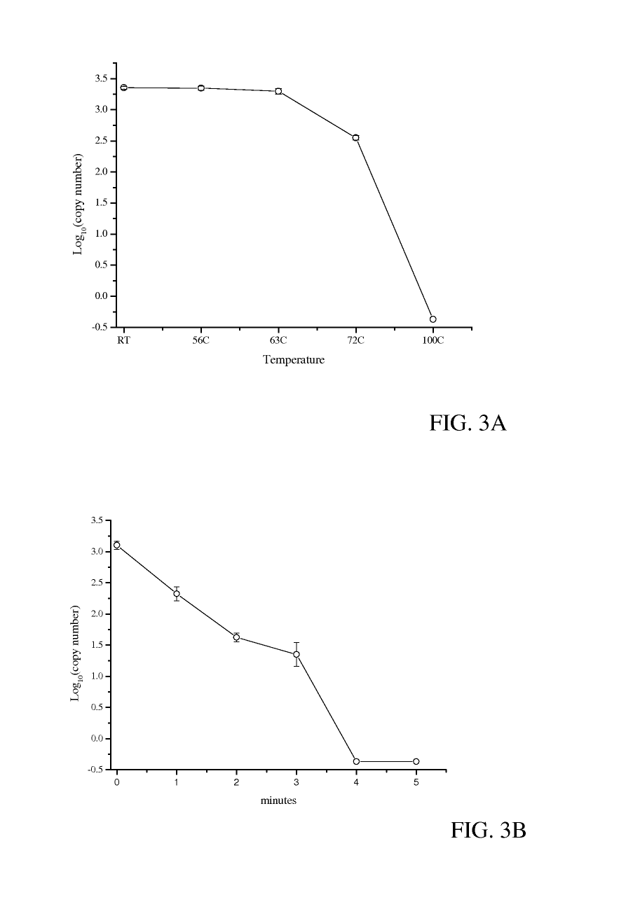

HuNoV is more resistant to heat than its surrogates. When HuNoV was exposed for 2 min to room temperature, 56, 63, 72, and 100° C., the virus copy number (Log 10) measured by ISC-qRT-PCR was 3.25 (±0.02), 3.35 (±0.03), 3.30 (±0.05), 2.55 (±0.04), and −0.37 (±0), respectively. HuNoV could only be fully inactivated by heating at 100° C. for 2 min. Only 84.3% of HuNoV could be inactivated by heating at 72° C. for 2 min. When HuNoV was heated at 72° C. for 4 min, the virus could be fully inactivated (FIG. 3B). However, when the virus was heated at 63° C. for over 60 min, only 96.1% virus could be inactivated (FIG. 3C).

### Chlorine Inactivation of HuNoV

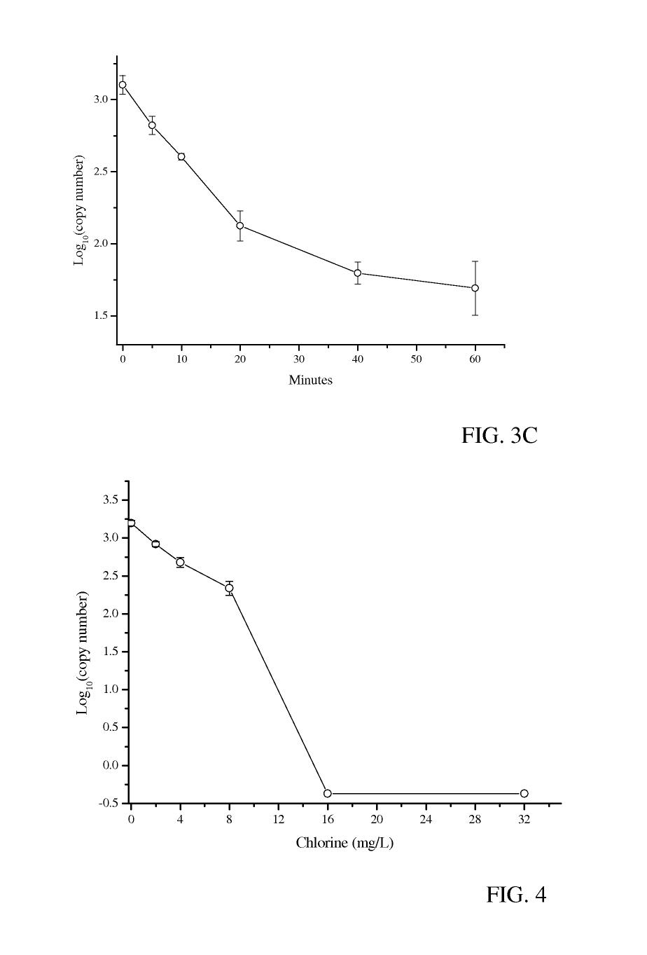

Relative to its surrogates, HuNoV is quite sensitive to chlorine treatment. The inactivation of chlorine was dose-dependent (FIG. 4). When HuNoV was treated with chlorine at concentrations of 0, 2, 4, and 8 for 10 min, the virus copy number (Log 10) measured by ISC-qRT-PCR was 3.20 (±0.04), 2.92 (±0.03), 2.68 (±0.07), and 2.34 (±0.09), respectively. When the virus was treated at a concentration of 16 ppm and above, HuNoV could be fully inactivated. In fact, HuNoV could be fully inactivated (no detectable Ct) in as short as 20 s when treated at 16 ppm chlorine (data not shown).

### UV Irradiation Inactivation of HuNoV.

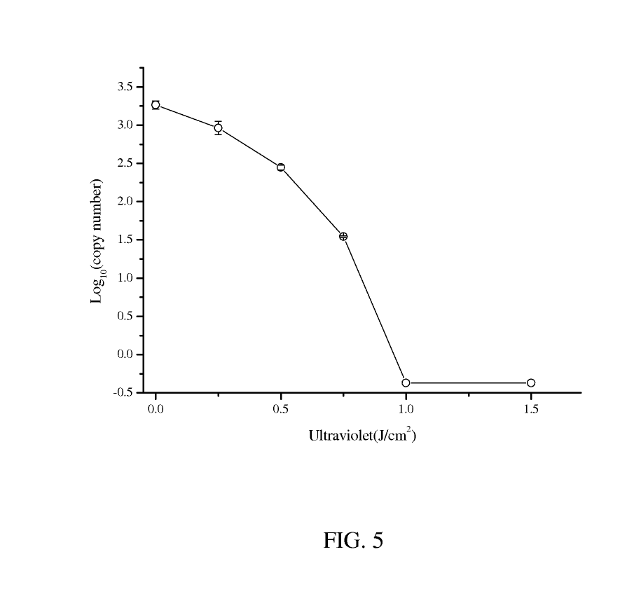

There was a dose-dependent inactivation of HuNoV measured by ISC-qRT-PCR (FIG. 5). When HuNoV were exposed to UV radiation at a dose of 0, 250, 500, 750, 1000, and 1500 mJ/cm2, the virus copy number (Log 10) measured by ISC-qRT-PCR was 3.26 (±0.05), 2.96 (±0.09), 2.45 (±0.03), 1.54 (±0.01), −0.37 (±0), and −0.37 (±0), respectively. The virus could be fully-inactivated at a UV dose of 1 K mJ/cm2.

### EtOH Inactivation of HuNoV.

EtOH was observed to have a limited effect for inactivating HuNoV. When HuNoV was treated by 70% EtOH, the signal from captured viral RNA was dramatically reduced (Table 1), the reduction in copy number being 99.33%. However, in five independent experiments (N=5) in triplicate (n=3), a clear amplification signal could still be detected in some samples, indicating that inactivation of HuNoV by 70% EtOH was not complete. When the viral concentration was increased 10 times, the reduction in copy number decreased to 88.5%. True amplification signals could be detected in almost all samples (data not shown).

TABLE 1

| | Ct | ΔCt | Reduction(%) |
|---|---|---|---|
| Positive control | 31 ± 0.9 | 9.7 | 99.3 ± 0.8 |
| 70% EtOH | 40.7 ± 1.4 | | |

### Discussion for Example 2

Evidence for the efficacy of disinfection agents against HuNoV have been based on studies using culturable surrogates such as FCV, MNV, and TV. However, surrogates and HuNoV exhibit different biological and physiochemical properties. The inactivation conditions for surrogates might not correctly reflect the inactivation conditions for HuNoV. Even among the surrogates, the inactivation conditions are different. FCV could be completely inactivated at 56° C. for 8 min and 71.3° C. for 1 min (Duizer et al., (2004) Appl Environ Microbiol 70(8), 4538-43) or at 50° C. for 30 min (Studdert, Martin, and Peterson, (1970) Am J Vet Res 31(10), 1723-32). MNV could be completely inactivated at 63° C. for 5 min or 72° C. for 1 min (Hewitt, Rivera-Aban, and Greening, (2009) J Appl Microbiol 107(1), 65-71). Complete inactivation was observed when TV was incubated at 63° C. and 72° C. for 5 min (Tian et al., 2013, supra). Based upon studies of thermal inactivation on surrogates, it has been suggested that a higher virus inactivation efficiency may be expected from regular batch (63° C. for 30 min) or classical pasteurization (72° C. for 2 min) than short time pasteurization (72° C. for 15 s) (Duizer et al., 2004, supra; J. Joukje Siebenga, (2010) “Norovirus Epidemiology, 5-7. In: Grant S. Hansman, Xi Jason Jiang and Kim Y. Green. Caliciviruses: Molecular and Cellular Virology.” Caister Academic Press). However, our study indicates that these conditions could not fully inactivate HuNoV. HuNoV is more heat-resistant than its surrogates. A complete inactivation (greater than 3 logs of reduction in copy number) was only observed when HuNoV was incubated at 72° C. for 4 min. Even incubation at 63° C. for 60 min, could not be fully inactivate HuNoV.

In current practice for the processing of leafy greens, chlorine (up to 200 ppm) is the most commonly-used sanitizer for washing fresh produce. Chlorine solution is not effective (usually less than 1.2-log virus reduction) in removing viral contaminants from fresh produce, base upon studies using surrogates (Bae and Schwab, (2008) Appl Environ Microbiol 74(2), 477-84; Baert et al., (2009) Food Prot 72(5), 1047-54; Dawson et al., (2005) J Appl Microbiol 98(1), 203-9). The difference in chlorine resistance varies even more than that of thermal inactivation resistance among HuNoV and its surrogates. A 2-log reduction in infectivity was observed when FCV was treated with 100 ppm chlorine for 1 min (Whitehead and McCue, (2009) Am J Infect Control 38(1), 26-30). However, the observed conditions for complete inactivation of FCV varies from 1,000 ppm for 1 min (Whitehead and McCue, 2009, supra), 5,000 ppm for 1.9 min (Park and Sobsey, 2011 Foodborne Pathog Dis 8(9), 1005-10) to 2,500-3,000 ppm for 10 min (Di Martino et al., 2010 Arch Virol 155(12), 2047-51; Duizer et al., 2004, supra). MNV could be inactivated by chlorine from 2,600 ppm for 20 sec (Belliot et al., 2008 Appl Environ Microbiol 74(10), 3315-8) to 5,000 ppm for 3.2 min (Park, Linden, and Sobsey, 2011, supra). TV is less resistant to chlorine than FCV and MNV. Three logs of reduction in titer was observed when TV was treated with 500 ppm of free chlorine (Tian et al., 2013, supra). The only human volunteer study demonstrated that HuNoV could not be inactivated by 3.75-6.25 mg/L chlorine, a concentration used for sanitization of drinking water (Keswick et al., 1985). Employing a molecular approach like traditional RT-PCR, Duizer et al., reported that HuNoVs could be inactivated by exposing the viruses to 6,000 ppm chlorine for 10 min (Duizer et al., 2004, supra). Therefore, HuNoVs are believed to be more resistant to chlorine than other GI viruses (Karst et al., 2003 Science 299(5612), 1575-8). Based upon FCV data, the U.S. Environmental Protection Agency suggests a chlorine bleach solution with a concentration of 1,000-5,000 ppm as being effective against HuNoV. In our study, we demonstrated that HuNoV is very sensitive to chlorine treatment. The virus could be fully inactivated by as little as 16 ppm of free chlorine in just 20 seconds. The difference in chlorine sensitivity between HuNoV and its surrogates might derive from differences in viral structure and/or differences in buffer systems used for testing. Most data from surrogates are from virus diluted in culture media, which contains nutrients such as amino acids and proteins. The free amino acids present in the media might bind or neutralize the free chlorine before it reaches the virus and reduces the effect of chlorine on virus. In fact, when HuNoV was diluted in HyClone M199 media (a common media for cultivating TV), there was no significantly effect of chlorine on HuNoV treated with 300 ppm of free chlorine (data not shown).

There is no direct data indicting the effectiveness of EtOH at inactivating HuNoV. All surrogates have been demonstrated sensitive to 70% EtOH. To exceed 4 D reduction in virus titer in 30 seconds, FCV required 70% EtOH (Gehrke, Steinmann, and Goroncy-Bermes, 2004 J Hosp Infect 56(1), 49-55), MNV required 60% EtOH (Belliot et al., 2008, supra), and TV required 50-70% EtOH (Tian et al., 2013, supra). However, EtOH is not as effective on HuNoV as its surrogates. Clearly, the effect of EtOH on HuNoV is related to virus concentration tested, as EtOH had a greater effect on HuNoV when HuNoV titer was low. The average probability of infection for a single norovirus particle is close to 0.5 based upon volunteer study and mathematical modeling (Teunis et al., 2008). In our study, even at a low titer of HuNoV, 70% EtOH did not result in a 3 log reduction in copy numbers. When the low HuNoV titer was increased 10 times, the reduction in virus copy number dropped from 99.3% to 88.5% (this study). Considering HuNoV's high infectivity, 70% EtOH only has a limited effect on disinfection of HuNoV.

FCV could be completely inactivated by UV-C between energy doses of 12 and 34 mJ/cm2 (De Roda Husman et al., 2004 Appl Environ Microbiol 70(9), 5089-93; Duizer et al., 2004, supra; Park, Linden, and Sobsey, 2011, supra) and MNV could be inactivated by UV-C between energy doses between of 25 and 27 mJ/cm2 (Lee, Zoh, and Ko, 2008 Appl Environ Microbiol 74(7), 2111-7; Park, Linden, and Sobsey, 2011, supra). With an inactivation dose of 60 mJ/cm2, TV is seemingly more UV-resistant than FCV and MNV (Tian et al., 2013, supra). In this study, we demonstrated a dose-dependent loss of the captured viral RNA when the radiation dosage was increased. The captured viral genomic signal was completely lost when HuNoV was treated at a dose above 1 J/cm². Dancho et al., reported that the required UV energy dosage for complete inactivation of HuNoVs could be as high as 2 J/cm² with PGM-conjugated magnetic beads based capture assay (Dancho, Chen, and Kingsley, 2012 Int J Food Microbiol 155(3), 222-6). However, for full-inactivation of HuNoV as determined be ISC-qRT_PCR, this dosage seems to be an overestimate. In the TV model, we demonstrated that TV could be fully inactivated at a dose of 60 mJ/cm², however, the ΔCt between treatment and control was only 0.75, reflecting a reduction of 71.5% in viral copy numbers. According to this model, a radiation dosage of 250 mJ/cm² resulted in a ΔCt of 1.35, suggesting full-inactivation of HuNoV.

ISC-qRT-PCR is an improved method 1) to effectively recover multiple strains of HuNoV from clinical, environmental and food samples, 2) minimize RT-PCR inhibitors present in clinical, environmental and food samples, and 3) a means of determining the infectivity of HuNoV. For our knowledge, this is the first report on inactivation conditions for HuNoV. Being an in situ assay makes it faster and easier. There was no need to extract viral RNA or to transfer the captured virus from magnetic beads to PCR tubes for further amplification. Therefore, ISC-qRT-PCR can be easily adapted for automated systems for processing of multiple samples. However, using this method to evaluate inactivation of HuNoV has some limitations. The ISC-qRT-PCR method could be used to evaluate virus inactivation deriving from capsid damage, and study interactions between the capsid and viral receptor. Heat, chlorine, and EtOH treatment primarily affect the capsid structure, which in turns affects the ability for the capsid to bind to viral receptors. Therefore, inactivation of HuNoV by these methods could be correctly reflected by ISC-qRT-PCR method. However, the mechanism of virus inactivation by UV radiation is entirely different. We demonstrated in the TV model that a lower dose of UV could damage the viral RNA genome, but does not change the binding of virus to the receptor. Therefore, this mechanism of inactivation can only be measured by a nucleic-acid-amplification-based assay.

It is understood that the examples and embodiments described herein are for illustrative purposes only and that various modifications or changes in light thereof will be suggested to persons skilled in the art and are to be included within the spirit and purview of this application and scope of the appended claims.

What is claimed is:

1. A method for detecting infectious capsid-enveloped viral particles in a biological sample, the method comprising:
(i) attaching a viral receptor to a container substrate thereby providing a container affixed capture agent;
(ii) adding the biological sample to the container affixed capture agent;
(iii) incubating the biological sample with the container affixed capture agent thereby capturing infectious capsid-enveloped viral particles as receptor-binding-intact capsids and providing receptor-bound-intact-capsids, wherein the receptor-bound-intact-capsids contain viral genomic nucleic acids;
(iv) adding PCR reagents to the receptor-bound-intact-capsids;
(v) amplifying the viral genomic nucleic acids contained within the receptor-bound-intact capsids with the PCR reagents, thereby providing amplified viral genomic nucleic acids as PCR products;
(vi) analyzing the amplified viral genomic nucleic acids to determine the presence and quantity of infectious capsid-enveloped viral particles;
thereby detecting infectious capsid-enveloped viral particles in a biological sample.

2. The method of claim 1, wherein the capsid-enveloped viral particles are members selected from the group consisting of human Norovirus and Tulane virus.

3. The method of claim 1, wherein the receptor is a member selected from the group consisting of Human Blood Group Antigen (HBGA), porcine gastric mucin (PGM) and specific antibodies.

4. A method for detecting infectious human Norovirus (HuNoV) in a biological sample, the method comprising:
(i) attaching an HuNoV receptor to a container substrate thereby providing a container affixed capture agent;
(ii) adding the biological sample to the container affixed capture agent;
(iii) incubating the biological sample with the container affixed capture agent thereby capturing infectious HuNoV as receptor-binding-intact capsids and providing receptor-bound-intact-capsids, wherein the receptor-bound-intact-capsids contain HuNoV genomic nucleic acids;
(iv) adding PCR reagents to the receptor-bound-intact-capsids;
(v) amplifying the HuNoV genomic nucleic acids contained within the receptor-bound-intact capsids with the PCR reagents, thereby providing amplified HuNoV genomic nucleic acids as PCR products;
(vi) analyzing the amplified HuNoV genomic nucleic acids to determine the presence and quantity of infectious HuNoV;
thereby detecting infectious HuNoV in a biological sample.

5. The method of claim 4, wherein the biological sample is a member selected from the group consisting of a clinical sample, an environmental sample and a food sample or a combination of said members.

6. The method of claim 4, wherein the HuNoV receptor is a member selected from the group consisting of Human Blood Group Antigen (HBGA) and porcine gastric mucin (PGM).

7. The method of claim 4, wherein the container substrate is a microtiter plate.

8. The method of claim 4, wherein the method is used to confirm that a sample has been disinfected of HuNoV, the method comprising:
(a) carrying out steps (i)-(vi) on the sample prior to disinfection,
(b) carrying out steps (i)-(vi) on the sample after disinfection and
(c) comparing results of steps a and b above to determine a quantity of infectious capsid-enveloped particles in step b and step a;
(d) determining if the quantity of infectious capsid-enveloped particles in step b is less than the quantity of infectious capsid-enveloped particles in step a, and
(e) deciding that the sample has been disinfected of HuNoV when the quantity of infectious capsid-enveloped particles in step b is less than the quantity of infectious capsid-enveloped particles in step a.


SEQUENCE LISTING (7 sequences)

SEQ ID NO: 1 - DNA, 18 bp, Artificial Sequence
  Primer TV forward
tgacgatgaccttgcgtg

SEQ ID NO: 2 - DNA, 23 bp, Artificial Sequence
  TV reverse primer
tgggattcaaccatgatacagtc

SEQ ID NO: 3 - DNA, 22 bp, Artificial Sequence
  TV probe
accccaaagccccagagttgat

SEQ ID NO: 4 - DNA, 22 bp, Artificial Sequence
  Primer P885F
ttggctcaacactgtgcaaaag

SEQ ID NO: 5 - DNA, 22 bp, Artificial Sequence
  Primer P886R
ctgattgcattttccaaacagc

SEQ ID NO: 6 - DNA, 22 bp, Artificial Sequence
  Primer TV-ORF2F
atggaaaacagcaaaactgaac

SEQ ID NO: 7 - DNA, 22 bp, Artificial Sequence
  Primer TV-ORF2R
ttatctaaagacaactgctgtc